(19) United States
(12) Patent Application Publication
(10) Pub. No.: US 2009/0149688 A1
(43) Pub. Date: Jun. 11, 2009

(54) Purging of regenerated adsorbent from an oxygenate removal unit

(75) Inventor: Jason T. Corradi, Arlington Heights, IL (US)

Correspondence Address: HONEYWELL INTERNATIONAL INC;PATENT SERVICES, 101 COLUMBIA DRIVE, P O BOX 2245 MAIL STOP AB/2B, MORRISTOWN, NJ, 07962, US

(73) Assignee: UOP LLC, a corporation of the State of Delaware, Des Plaines, IL (US)

(21) Appl. No.: 12/001,272

(22) Filed: Dec. 11, 2007

(51) Int. Cl. C07C 7/12

(52) U.S. Cl. 585/826

(57) ABSTRACT

The processes and systems disclosed herein relate to the removal of contaminants from the effluent of an oxygenate removal unit (ORU). More particularly, the processes and systems disclosed herein relate to the removal of contaminants resulting from the regeneration of adsorbers in an ORU. Processes and systems are provided for regeneration of an adsorber in an oxygenate removal unit comprising: (a) providing an oxygenate removal unit comprising at least one adsorber, wherein the at least one adsorber comprises a feed end and an effluent end; (b) passing a liquid hydrocarbon feedstock to the feed end of the at least one adsorber and removing an effluent stream from the effluent end of the adsorber; (c) isolating the at least one adsorber for regeneration by terminating passage of the liquid hydrocarbon feedstock to the feed end of the adsorber; (d) removing substantially all of the effluent stream from the adsorber; (e) regenerating the adsorber with a regeneration gas; (f) refilling the adsorber with an inventory liquid; and (g) purging the regenerated adsorber with a slipstream of liquid hydrocarbon feedstock to displace the inventory liquid.

### TECHNICAL FIELD

The processes and systems disclosed herein relate to the removal of contaminants from the effluent of an Oxygenate Removal Unit (ORU). More particularly, the processes and systems disclosed herein relate to the removal of contaminants resulting from the regeneration of adsorbent in an ORU.

### BACKGROUND

Light olefins and other related hydrocarbons serve as feeds for the production of numerous chemicals. Light olefins have traditionally been produced from petroleum sources. However, oxygenates such as alcohols, particularly methanol, ethanol, and higher alcohols or their derivatives, are used as alternative materials for light olefin production. These alcohols may be produced by fermentation or from synthesis gas. Oxygenates are particularly attractive because they can be produced from such widely available materials as coal, natural gas, recycled plastics, various carbon waste streams from industry and various products and by-products from the agricultural industry.

Although many oxygenates have been discussed in the prior art, the principal focus on producing the desired light olefins has been on methanol conversion technology, primarily because of the availability of commercially proven methanol synthesis technology. Various methanol to olefin (MTO) procedures for catalytically converting methanol into the desired light olefin products have been developed.

The product stream from MTO process is generally a raw product stream containing impurities. For example, a product stream from an MTO process typically contains light olefins, oxygenates, and water. The product stream undergoes a process to remove the impurities and separate the light olefins.

### SUMMARY

The processes and systems disclosed herein relate to the removal of impurities and separation the light olefins from an MTO product stream. Specifically, Oxygenate Removal Units (ORUs) are often incorporated into processes and systems for the treatment of MTO product streams to remove oxygenates. The processes and systems disclosed herein relate to the removal of contaminants from the effluent of an ORU. More particularly, the processes ad systems disclosed herein relate to the removal of contaminants resulting from the regeneration of adsorbers in an ORU.

In one aspect, process is provided for regeneration of an adsorber in an oxygenate removal unit comprising: (a) providing an oxygenate removal unit comprising at least one adsorber, wherein the at least one adsorber comprises a feed end and an effluent end; (b) passing a liquid hydrocarbon feedstock to the feed end of the at least one adsorber and removing an effluent stream from the effluent end of the adsorber; (c) isolating the at least one adsorber for regeneration by terminating passage of the liquid hydrocarbon feedstock to the feed end of the adsorber; (d) removing substantially all of the effluent stream from the adsorber; (e) regenerating the adsorber with a regeneration gas; (f) refilling the adsorber with an inventory liquid; and (g) purging the regenerated adsorber with a slipstream of liquid hydrocarbon feedstock to displace the inventory liquid. In at least one embodiment, the process further includes (h) transferring the displaced inventory liquid to an upstream operation unit that is upstream of the oxygenate removal unit.

In another aspect, a system is provided for regeneration of an adsorber in an oxygenate removal unit comprising: (a) an oxygenate removal unit comprising at least one adsorber, wherein the at least one adsorber comprises a feed end and an effluent end; (b) a supply of a liquid hydrocarbon feedstock that is fed to the feed end of the at least one adsorber; (c) an effluent stream that is removed from the effluent end of the adsorber; (d) a device that isolates the first adsorber from the system by terminating passage of the liquid hydrocarbon feedstock to the feed end of the first adsorber; (e) an effluent line operatively connected to the effluent end of the first adsorber that provides for removal of the effluent stream from the effluent end of the first adsorber; (f) a regeneration gas supply operatively connected to the adsorber that supplies regeneration gas to regenerate the adsorber; (g) an inventory liquid supply operatively connected to the adsorber that refills the adsorber with an inventory liquid; and (h) a slipstream supply operatively connected to the adsorber that supplies a slipstream of hydrocarbon feedstock to displace the inventory liquid.

### BRIEF DESCRIPTION OF SEVERAL VIEWS OF THE DRAWINGS

FIG. 1 is a representative process for regeneration of an adsorber in an oxygenate removal unit.

FIG. 2 is a representative process for regeneration of an adsorber in an oxygenate removal unit.

FIG. 3 is a representative process for regeneration of an adsorber in an oxygenate removal unit.

FIG. 4 is a representative system for regeneration of an adsorber in an oxygenate removal unit.

### DETAILED DESCRIPTION

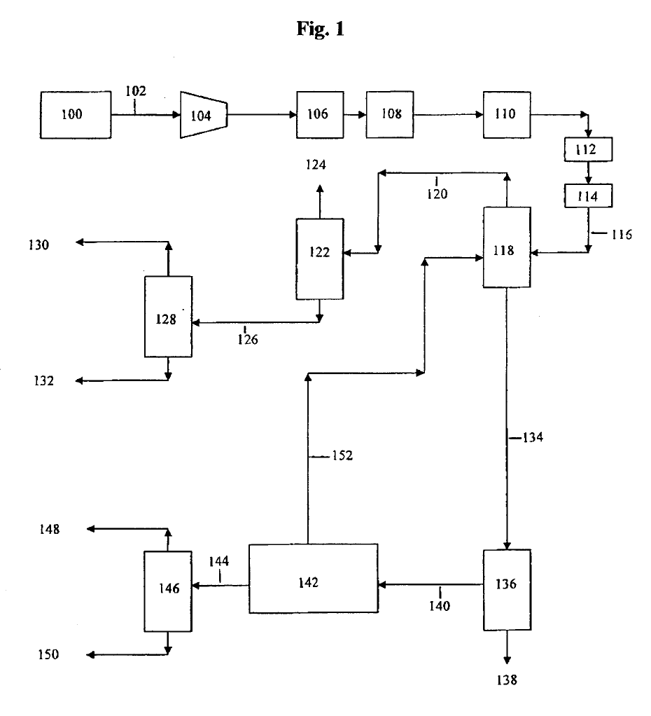

One example of a process for the removal of impurities and the separation of light olefins from an MTO product vapor stream is illustrated in FIG. 1. An MTO product vapor stream typically contains light olefins, oxygenates, and water. For example, an MTO product vapor stream can contain unreacted methanol, dimethyl ether intermediate, ethylene, propylene, C₄ to C₆ olefins, and minor amounts of other hydrocarbons and oxygenates. As illustrated, the product vapor stream 102 from the MTO process goes from the MTO process 100 to a compressor 104. The product vapor stream undergoes compression in the compressor 104, and the pressure of the vapor stream is increased. In at least some instances, liquid can be formed during compression, and is recycled upstream in the MTO process (not shown). The vapor stream exits the compressor 104 at increased pressure, and goes to an oxygenate absorber 106 and a scrubber 108. In the absorber 106, the vapor stream is contacted with a solvent such as, for example, water, to remove at least some oxygenates. In the scrubber 108, the vapor stream undergoes caustic scrubbing for bulk removal of carbon dioxide.

After undergoing caustic scrubbing, the vapor stream goes to a dryer 110, where moisture is removed from the vapor stream. The dried vapor stream then undergoes cooling 112 and is put through a distillation sequence 114 that results in the vapor stream becoming a liquid hydrocarbon feedstock 116. As further illustrated in FIG. 1, the liquid hydrocarbon feedstock goes to a deethanizer 118, which separates the C₁ and C₂ olefins from the olefins comprising C₃ or greater.

The feedstock 120 containing C₁ and C₂ hydrocarbons is sent from the deethanizer 118 to a demethanizer 122, where methane (C₁) 124 and other light impurities are removed. The resulting C₂ hydrocarbon feedstock 126 then goes to a C₂ splitter 128 that separates out ethylene 130 and ethane 132.

The feedstock 134 containing C₃ or greater hydrocarbons is sent from the deethanizer 118 to a depropanizer 136, where the C₃ fraction is removed from the remaining hydrocarbon feedstock 138 containing C₄ or greater hydrocarbons. The hydrocarbon feedstock 140 containing C₃ hydrocarbons goes to an oxygenate removal unit (ORU) 142. The oxygenate removal unit removes oxygenates such as, for example, dimethyl ether. The resulting product stream 144, sometimes referred to herein as the “effluent stream,” goes to a C₃ splitter 146, where propylene 148 and propane are separated 150.

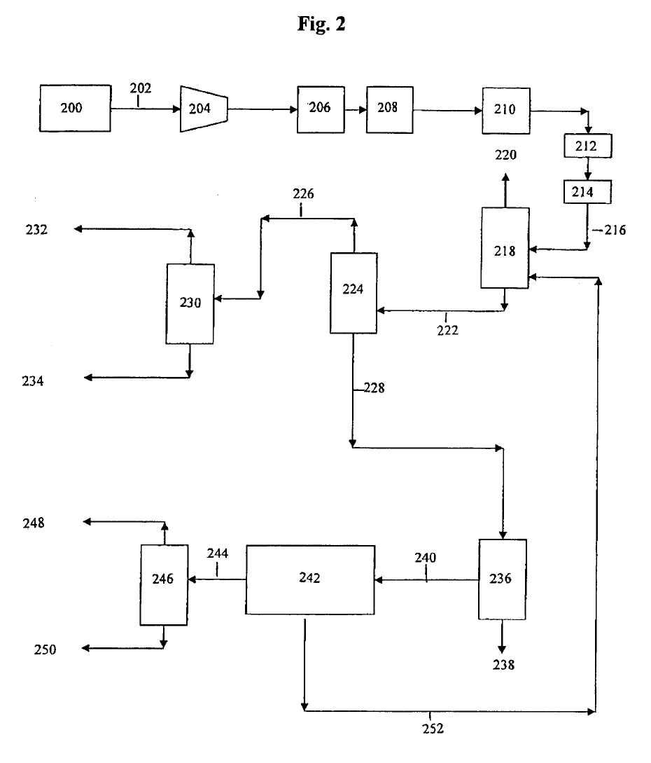

A second example of a process for the removal of impurities and the separation of light olefins from an MTO product vapor stream is illustrated in FIG. 2. This process is similar to the process illustrated in FIG. 1, in that an MTO product vapor stream 202 goes from the MTO process 200 to a compressor 204. The product vapor stream undergoes compression in the compressor 204, and the pressure of the vapor stream is increased. The vapor stream exits the compressor 204 at increased pressure, and goes to an oxygenate absorber 206 and a scrubber 208. In the absorber 206, the vapor stream is contacted with a solvent such as, for example, water, to remove at least some oxygenates. In the scrubber 208, the vapor stream undergoes caustic scrubbing for bulk removal of carbon dioxide.

After undergoing caustic scrubbing, the vapor stream goes to a dryer 210, where moisture is removed from the vapor stream. The dried vapor stream then undergoes cooling 212 and is put through a distillation sequence 214 that results in the vapor stream becoming a liquid hydrocarbon feedstock 216.

In contrast to FIG. 1, the liquid hydrocarbon feedstock 216 illustrated in FIG. 2 goes from the distillation sequence 214 to a demethanizer 218, which separates the C₁ hydrocarbons 220 from the remaining feedstock containing hydrocarbons comprising C₂ or greater 222.

The remaining feedstock 222 is passed from the demethanizer 218 to a deethanizer 224, where the ethane (C₂) fraction 226 is separated from the remaining feedstock containing hydrocarbons comprising C₃ or greater 228. The ethane fraction 226 is passed to an ethane/ethylene splitter column (C₂ splitter) 230 that separates out ethylene 232 and ethane 234.

The feedstock containing C₃ or greater hydrocarbons 228 is sent from the deethanizer 224 to a depropanizer 236, where the C₃ fraction is removed from the remaining hydrocarbon feedstock containing C₄ or greater olefins 238. The hydrocarbon feedstock containing the C₃ fraction 240 is passed to an oxygenate removal unit (ORU) 242. The oxygenate removal unit removes oxygenates such as, for example, dimethyl ether. The resulting product stream 244, sometimes referred to herein as the “effluent stream,” goes to a C₃ splitter 246, where propylene 248 and propane 250 are separated.

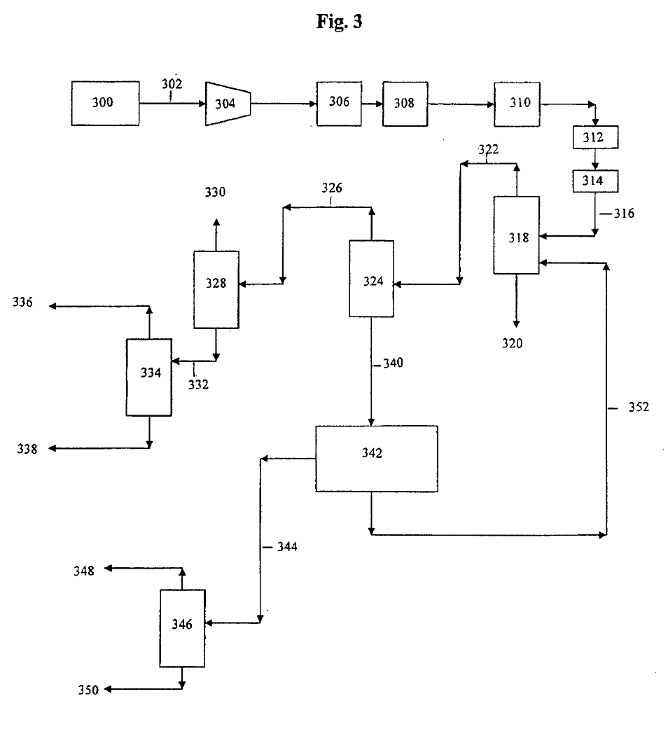

A third example of a process for the removal of impurities and the separation of light hydrocarbons from an MTO product vapor stream is illustrated in FIG. 3. This process is similar to the process illustrated in FIGS. 1 and 2, in that an MTO product vapor stream 302 goes from the MTO process 300 to a compressor 304. The product vapor stream undergoes compression in the compressor 304, and the pressure of the vapor stream is increased. The vapor stream exits the compressor 304 at increased pressure, and goes to an oxygenate absorber 306 and a scrubber 308. In the absorber 306, the vapor stream is contacted with a solvent such as, for example, water, to remove at least some oxygenates. In the scrubber 308, the vapor stream undergoes caustic scrubbing for bulk removal of carbon dioxide.

After undergoing caustic scrubbing, the vapor stream goes to a dryer 310, where moisture is removed from the vapor stream. The dried vapor stream then undergoes cooling 312 and is put through a distillation sequence 314 that results in the vapor stream becoming a liquid hydrocarbon feedstock 316.

In contrast to FIGS. 1 and 2, the liquid hydrocarbon feedstock 316 illustrated in FIG. 3 goes from the distillation sequence 314 to a deproanizer 318, where the C₄ fraction 320, containing hydrocarbons of C₄ or greater, is removed from the remaining hydrocarbon feedstock 322, containing the hydrocarbons of C₃ or lighter.

The hydrocarbon feedstock 322 containing the hydrocarbons of C₃ or lighter is passed to a deethanizer 324, where the hydrocarbons containing C₂ or lighter are separated from the hydrocarbon feedstock containing the C₃ hydrocarbons. The feedstock containing the C₂ or lighter olefins 326 is passed to a demethanizer 328, which separates the C₁ hydrocarbons 330 from the remaining C₂ hydrocarbons feedstock 332. The C₂ hydrocarbons feedstock 332 is passed to an ethane/ethylene splitter column (C₂ splitter) 334 that separates out ethylene 336 and ethane 338.

The hydrocarbon feedstock 340 containing the C₃ hydrocarbons is passed to an oxygenate removal unit (ORU) 342. The oxygenate removal unit 342 removes oxygenates such as, for example, dimethyl ether. The resulting product stream 344, sometimes referred to herein as the “effluent stream,” goes to a propylene/propane splitter column (C₃ splitter) 346, where propylene 348 and propane are separated 350.

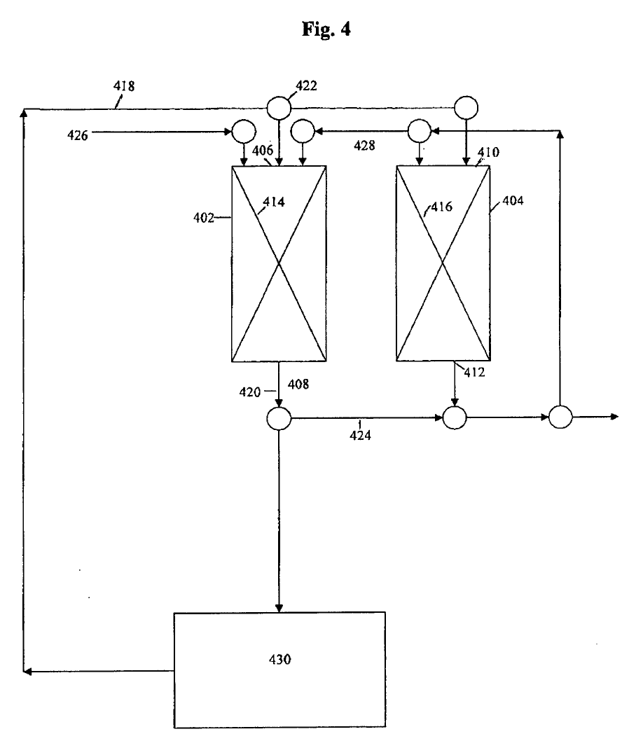

In the processes and systems disclosed herein, the oxygenate removal unit (ORU) has at least one adsorber, and preferably has a plurality of adsorbers, to remove oxygenates. FIG. 4 illustrates a particularly preferred system for regeneration of an adsorber in accordance with the process described above. As illustrated in FIG. 4, the ORU has a first adsorber 402, a second adsorber 404. Each adsorber has a feed end and an effluent end. For example, as illustrated in FIG. 4, first adsorber 402 has feed end 406 and effluent end 408, and second adsorber 404 has feed end 410 and effluent end 412. Additionally, each adsorber includes an adsorbent bed that contains a solid adsorbent capable of selectively adsorbing trace amounts of oxygenates. For example, the first adsorber 402 has adsorbent bed 414, the second adsorber 404 has adsorbent bed 416.

A supply of a liquid hydrocarbon feedstock is fed to the feed end of at least one adsorber. As illustrated in FIG. 4, a feedstock line 418 passes a liquid hydrocarbon feedstock to the feed end 406 of adsorber 402. An effluent stream is removed from the effluent end of at least the first adsorber through outlet piping 420. In preferred processes and systems, the liquid hydrocarbon feedstock is a product stream from an MTO process that contains propylene.

Adsorbers require regular, independent regeneration. Regeneration of one adsorber can begin by isolating, for example, first adsorber 402 for regeneration by terminating passage of the liquid hydrocarbon feedstock to the feed end 406 of the adsorber. There is a device 422 that isolates the first adsorber from the system by terminating passage of the liquid hydrocarbon feedstock to the feed end 406 of the first adsorber 402. The device 422 may be a valve that can be closed to prevent the flow of liquid hydrocarbon feedstock into the first adsorber.

Preferably, once the first adsorber 402 is isolated, it is drained by removing substantially all of the effluent stream from the adsorber. An effluent line 424 operatively connected to the effluent end of the first adsorber that provides for removal of the effluent stream from the effluent end of the first adsorber. It is particularly preferred that substantially all of the removed effluent stream be transferred from the adsorber 402 to another adsorber such as, for example, adsorber 404. The term “substantially all” is used in this context to indicate that a residual amount of effluent stream tends to remain within the first adsorber, as well as within the outlet piping at the effluent end of the first adsorber. The adsorber to which the effluent stream, is transferred is preferably an adsorber that has undergone regeneration immediately prior to receiving the effluent stream from the first adsorber 402, and is in the process of coming back on-stream. The effluent stream from the first adsorber 402 is preferably used to fill the adsorber coming back on-stream prior to re-initiating the flow of hydrocarbon feedstock the adsorber coming back on-stream.

After the removal of substantially all of the effluent stream from the adsorber that has been isolated for regeneration, the adsorber can be regenerated with a regeneration gas. There is a regeneration gas supply 426 operatively connected to the adsorber that supplies regeneration gas to regenerate the adsorber. In at least some instances, nitrogen or methane is utilized as the regeneration gas. Preferably, in some instances, the overhead distillate vapor from the upstream demethanizer is utilized as the regeneration gas. The overhead distillate vapor from the upstream demethanizer, however, can be contaminated with light olefins such as, for example, methane and ethylene.

In preferred processes, the regeneration gas is passed to the adsorbent bed of the adsorber at a temperature effective to desorb oxygenates from the solid adsorbent and recover the oxygenates from the adsorbent bed in a spent regenerant vapor stream. Upon regeneration of the adsorber with the regeneration gas, at least some residual regeneration gas tends to remain in the adsorber.

After regeneration of the adsorber with the regeneration gas, the regenerated adsorber is refilled with an inventory liquid. Accordingly, the system illustrated in FIG. 4 has an inventory liquid supply 428 operatively connected to the adsorber that refills the adsorber with an inventory liquid. The inventory liquid preferably comprises hydrocarbons, such as propylene. More preferably, the inventory liquid is effluent stream that has been removed from another adsorber such as, for example, second adsorber 404, that is beginning to undergo regeneration as described herein with respect to the first adsorber 402.

As the adsorber is refilled, residual regeneration gas is absorbed into the inventory liquid. Any contaminates in the residual regeneration gas are thus also absorbed into the inventory liquid. It is preferred that the regenerated adsorber be purged with a slipstream to displace the inventory liquid. The slipstream is preferably liquid hydrocarbon feedstock. Accordingly, a slipstream supply is preferably operatively connected to the adsorber that supplies a slipstream of hydrocarbon feedstock to displace the inventory liquid. As illustrated in FIG. 4, the slipstream supply is preferably the hydrocarbon feedstock supply 418. The displaced inventory liquid is then preferably transferred to an upstream operation unit 430, and the regenerated gas, as well as the contaminates therein, that dissolved in the inventory liquid during refilling of the adsorber can be removed as the inventory liquid proceeds through the system from the upstream unit. If not displaced and transferred upstream, the inventory liquid would be passed to the propylene/propane splitter as effluent stream, thus contaminating the propylene and/or propane products. In order to avoid having a significant affect on the upstream operation unit, the slipstream is from about 10% to about 20% of the volume of influent to the upstream operation unit from any other process steps.

For example, with respect to the process illustrated in FIG. 1, the displaced inventory liquid 152 is transferred from the oxygenate removal unit 142 to the deethanizer 118. To accomplish this transfer, there is preferably a transfer line operatively connected to the regenerated adsorber to transfer the displaced inventory liquid to the deethanizer.

As another example, with respect to the process illustrated in FIG. 2, the displaced inventory liquid 252 is transferred from the oxygenate removal unit 242 to the demethanizer 218. To accomplish this transfer, there is preferably a transfer line operatively connected to the regenerated adsorber to transfer the displaced inventory liquid to the demethanizer.

As a third example, with respect to the process illustrated in FIG. 3, the displaced inventory liquid 352 is transferred from the oxygenate removal unit 342 to the depropanizer 318. To accomplish this transfer, there is preferably a transfer line operatively connected to the regenerated adsorber to transfer the displaced inventory liquid to the depropanizer.

From the foregoing, it will be appreciated that although specific representative structures and processes have been described herein for purposes of illustration, various modifications may be made without deviating from the spirit or scope of the disclosure. It is therefore intended that the foregoing detailed description be regarded as illustrative rather than limiting, and that it be understood that it is the following claims, including all equivalents, that are intended to particularly point out and distinctly claim the disclosure subject matter.

What is claimed is:

1. A process for regeneration of an adsorber in an oxygenate removal unit comprising:
(a) providing an oxygenate removal unit comprising at least one adsorber, wherein the at least one adsorber comprises a feed end and an effluent end;
(b) passing a liquid hydrocarbon feedstock to the feed end of the at least one adsorber and removing an effluent stream from the effluent end of the adsorber;
(c) isolating the at least one adsorber for regeneration by terminating passage of the liquid hydrocarbon feedstock to the feed end of the adsorber;
(d) removing substantially all of the effluent stream from the adsorber;
(e) regenerating the adsorber with a regeneration gas;
(f) refilling the adsorber with an inventory liquid; and
(g) purging the regenerated adsorber with a slipstream of liquid hydrocarbon feedstock to displace the inventory liquid.

2. The process of claim 1, further comprising transferring the displaced inventory liquid to an upstream operation unit.

3. The process of claim 2, wherein the upstream operation unit is a deethanizer, a demethanizer, or a depropanizer.

4. The process of claim 1, further comprising removing regenerated gas dissolved in the inventory liquid during the step of (f) refilling the adsorber with an inventory liquid.

5. The process of claim 1, wherein the slipstream is from about 10% to about 20% of the volume of influent to the upstream operation unit.

6. The process of claim 1, wherein the regeneration gas is nitrogen or methane.

7. The process of claim 1, wherein the inventory liquid comprises hydrocarbons.

8. The process of claim 1, wherein the inventory liquid comprises propylene.

9. The process of claim 1, wherein the slipstream comprises liquid hydrocarbon feedstock.

10. The process of claim 1, wherein the oxygenate removal unit comprises a plurality of adsorbers, wherein each adsorber comprises a feed end and an effluent end.

11. A process for regeneration of an adsorber in an oxygenate removal unit comprising:
(a) providing an oxygenate removal unit comprising at least one adsorber, wherein the at least one adsorber comprises a feed end and an effluent end;
(b) passing a liquid hydrocarbon feedstock to the feed end of the at least one adsorber and removing an effluent stream from the effluent end of the adsorber;
(c) isolating the at least one adsorber for regeneration by terminating passage of the liquid hydrocarbon feedstock to the feed end of the adsorber;
(d) removing substantially all of the effluent stream from the adsorber;
(e) regenerating the adsorber with a regeneration gas;
(f) refilling the adsorber with an inventory liquid;
(g) purging the regenerated adsorber with a slipstream of liquid hydrocarbon feedstock to displace the inventory liquid; and
(h) transferring the displaced inventory liquid to an upstream operation unit upstream of the oxygenate removal unit.

12. The process of claim 11, wherein the upstream operation unit is a deethanizer, a demethanizer, or a depropanizer.

13. The process of claim 11, further comprising removing regeneration gas dissolved in the inventory liquid during the step of (f) refilling the adsorber with an inventory liquid.

14. The process of claim 11, wherein the slipstream is from about 10% to about 20% of the volume of influent to the upstream operation unit.

15. The process of claim 11, wherein the regeneration gas is nitrogen or methane.

16. The process of claim 11, wherein the inventory liquid comprises propylene.

17. The process of claim 11, wherein the slipstream comprises liquid hydrocarbon feedstock.

18. A system for regeneration of an adsorber in an oxygenate removal unit comprising:
(a) an oxygenate removal unit comprising at least one adsorber, wherein the at least one adsorber comprises a feed end and an effluent end;
(b) a supply of a liquid hydrocarbon feedstock that is fed to the feed end of the at least one adsorber;
(c) an effluent stream that is removed from the effluent end of the adsorber;
(d) a device that isolates the first adsorber from the system by terminating passage of the liquid hydrocarbon feedstock to the feed end of the first adsorber;
(e) an effluent line operatively connected to the effluent end of the first adsorber that provides for removal of the effluent stream from the effluent end of the first adsorber;
(f) a regeneration gas supply operatively connected to the adsorber that supplies regeneration gas to regenerate the adsorber;
(g) an inventory liquid supply operatively connected to the adsorber that refills the adsorber with an inventory liquid; and
(h) a slipstream supply operatively connected to the adsorber that supplies a slipstream of hydrocarbon feedstock to displace the inventory liquid.

19. The system of claim 18, further comprising an upstream operation unit and a transfer line operatively connected to the adsorber to transfer the displaced inventory liquid to the upstream operation unit.

20. The system of claim 19, wherein the slipstream is from about 10% to about 20% of the volume of influent to the upstream operation unit.
